(19) United States
(12) Patent Application Publication
(10) Pub. No.: US 2024/0296457 A1
(43) Pub. Date: Sep. 5, 2024

(54) SYSTEMS AND METHODS FOR ACCESSING MULTI-TENANT POINT-OF-SERVICE FUNCTIONALITIES ASSOCIATED WITH A SPECIFIC LOCATION

(71) Applicant: Ricardo Cabrera, Miami, FL (US)

(72) Inventor: Ricardo Cabrera, Miami, FL (US)

(21) Appl. No.: 18/661,581

(22) Filed: May 11, 2024

Related U.S. Application Data
(63) Continuation-in-part of application No. 17/860,273, filed on Jul. 8, 2022.

(51) Int. Cl. G06Q 30/015; G16H 40/20

(52) U.S. Cl. CPC G06Q 30/015; G16H 40/20

(57) ABSTRACT

A computer-implemented method for managing visitor access at a business facility that houses multiple tenants is described. Each tenant has their tenant-specific access to a shared computer system, allowing them to set up specific entry point and exit point functionalities for their visitors. Visitor interaction is done via a front desk access panel accessible to the visitor upon entry to the business location. When a visitor arrives and self-identifies themselves at this access panel, the computer system verifies them as a visitor to a particular tenant. The visitor can then use the panel to access specific functionalities designed for entering or moving within the tenant's area. When the visitor's time at the tenant's location ends, the same access panel provides functionalities for a proper exit. This setup streamlines the process of visitor management, ensuring each visitor has access only to relevant areas based on which tenant they are visiting.

### CROSS-REFERENCE DATA

This US patent application claims a priority date benefit from a co-pending U.S. patent application Ser. No. 17/860,273 filed 8 Jul. 2022 and entitled BUSINESS TRANSACTIONS AND WORKSPACE SHARING PLATFORM FOR MULTIPLE TENANTS, which in turn claims a priority date benefit from the U.S. Provisional patent application No. 63/349,198 filed 6 Jun. 2022 by the same inventor and with the same title, both patent documents are incorporated herein in their respective entireties by reference.

### BACKGROUND

Without limiting the scope of the invention, its background is described in connection with an automated cloud-based or local server-based software platform that allows multiple tenants housed at the same business location to reserve and use the shared workspace and its resources as well as perform a variety of business transactions with their customers and visitors. More particularly, the invention describes the method for accessing tenant-specific point-of-service visitor functionalities associated with at least one or more specific multi-tenant business locations.

Modern-day workspace in the post-Covid era is no longer a conventional office space occupied by the same company throughout the duration of the workday. With many companies switching to a fully remote or partially remote workforce, the need for office sharing is expected to increase significantly in the years to come. This is particularly true for many downtown locations in the business centers of major cities, where the cost of renting these facilities is high and constitutes a big expense line for many small businesses. One way for these businesses to remain in prestigious geographical locations is to share the cost of these workspaces with other businesses, thereby reducing their expenses and increasing their profitability.

In some cases, the tenants are present at the shared business location only on a part-time basis. At the same time, visitors, especially new or unannounced visitors may stop by at the business location at any time during business or even off-business hours. There is a need for the tenants to provide a comprehensive list of point-of-service functionalities for visitors that would not result in loosing a customer just because the tenant was not present at the business location at the time when a visitor stopped by.

In a shared business location with multiple tenants, a visitor typically follows a streamlined check-in process at a central front desk computer. Upon entering the building, the visitor approaches the front desk where a computerized check-in system is prominently placed. The system usually features a touchscreen interface for ease of use. The visitor begins the check-in process by selecting the option to check in, which prompts them to enter their name and the name of the tenant business they are visiting. This is often facilitated by a searchable database of tenant businesses housed in the building.

Once the visitor selects the specific tenant, they might be asked to type in the name of the person they are meeting. The system may then request additional details such as the purpose of the visit and a contact number. After entering all the required information, the visitor is typically prompted to take a photo using the built-in camera for a visitor badge that will be printed directly from the system. This badge usually includes their name, the date, and the tenant's name, serving as a pass to access certain areas of the building.

After the badge is issued, the system automatically notifies the host of their visitor's arrival through an internal communication, such as an email or a direct message to their company phone or computer. The visitor is then either instructed to wait in the lobby where their host will come to meet them, or they are given directions to proceed to the specific floor or office where the tenant business is located. Instructions or a map may be displayed on the screen or printed out for the visitor's convenience. This efficient process helps manage the flow of visitors and ensures security by keeping track of who enters and leaves the shared business space.

While these conventional systems at the front desk of a shared business facility offer a reasonably streamlined visitor management, they come with several drawbacks and limitations. Technical issues, such as system crashes or hardware malfunctions, can disrupt the check-in process and cause delays. Privacy is a significant concern, as visitors may be uneasy about their personal information, including photographs, being stored in a system with uncertain security measures. The lack of personal interaction due to automation can also detract from the visitor experience, potentially harming relationship-building in industries where personal connections are vital. Furthermore, the computer system interaction may be challenging for visitors who are not adept at using digital technologies, potentially leading to frustration and requiring additional assistance. Security vulnerabilities could arise if the system is accessed without authorization or if there is a data breach, posing risks to both visitor and tenant confidentiality. The absence of human oversight means potentially suspicious activities could go unnoticed, compromising security. Financially, the cost of installing, maintaining, and updating the system can be considerable, which might be a barrier for smaller entities. Lastly, accessibility could be an issue if the system does not adequately accommodate visitors with disabilities, thereby excluding some individuals from easy access to the facility. Thus, while the automated system aims to enhance efficiency and security, addressing these concerns are needed to ensure it is inclusive, secure, and beneficial for all stakeholders.

In addition, present systems do not provide the visitor with any tenant-specific exit point functionalities, such as payment for the visit, scheduling the next visit, etc. The need exists, therefore, for improved front desk systems to provide a more comprehensive visitor experience during both the initial and concluding interactions by the visitor.

### SUMMARY

Accordingly, it is an object of the present invention to overcome these and other drawbacks of the prior art by providing a novel method for accessing tenant-specific point-of-service visitor functionalities associated with a specific multi-tenant business location.

It is another object of the present invention to provide a novel method for sharing workspace and managing visitors between multiple tenants and their visitors in a single, easy-to-operate platform that is customizable for the needs of various small and medium-sized businesses.

It is a further object of the invention to provide the tenant's visitor with a customizable entry point and exit point tenant-specific functionalities corresponding to a specific business location.

It is a further object of the present invention to provide a novel method for accessing tenant-specific point-of-service visitor functionalities associated with a specific multi-tenant business location using a unique visitor-specific identifier.

It is a further object of the present invention to provide a novel method for accessing tenant-specific point-of-service visitor functionalities associated with a specific multi-tenant business location with improved security.

The computer-implemented method for accessing tenant-specific point-of-service visitor functionalities associated with a specific multi-tenant business location housing a plurality of business tenants may include the following steps:
a. providing each tenant with a tenant-specific access to a multi-tenant computer system to configure tenant-specific and location-specific visitor's functionalities for known and anticipated tenant's visitors to be operated using a front desk access panel at the specific business location,
b. upon arrival to the business location and self-identification of a visitor using the front desk access panel, operating the computer system to recognize and designate the visitor as a known tenant's visitor of a specific tenant at the business location,
c. operating the front desk access panel to allow the tenant's visitor to access the tenant-specific visitor's entry point functionalities associated with the tenant's business at the business location, and
d. upon completion of a visit with the tenant, operating the front desk access panel to provide the tenant's visitor with the tenant-specific exit point functionalities corresponding to the same business location.

The term “tenant” is used herein to describe an organization with contractual rights to the shared workspace. Each tenant may have a tenant ID, which can be used to identify the tenant in the novel software of the present invention. Each tenant may have one or more employees or contributors, each of whom may be assigned a distinct employee or personal ID associated with the tenant ID. Each entry point or exit point functionality performed by a visitor may involve automatically selecting a desired tenant as well as selecting desired employee or a person within the selected tenant organization.

In a further aspect of the present invention, the computer-implemented method may be further configured to be implemented in a multi-tenant or a single-tenant mode. While in either mode, it may be further configured to operate in a multi-person mode if a particular tenant has more than one employee or person associated therewith. One advantage of this flexibility is that when a tenant “graduates” from a workspace sharing arrangement to occupy their own workspace full time, they can continue to use the software based on the novel method of the present invention and operate in a single-tenant multi-person mode where individuals of that tenant and their respective visitors have the ability to performs appropriate functionalities using the same front desk tablet.

In a further aspect of the invention, computer-implemented method for accessing tenant-specific point-of-service visitor functionalities associated with a specific multi-tenant business location may include the following steps:
a. providing each tenant with a tenant-specific access to a multi-tenant computer system to configure tenant-specific and location-specific visitor's functionalities to be operated using a front desk access panel at the specific business location,
b. upon arrival and self-identification of a tenant's visitor to the specific business location, providing the tenant's visitor with the front desk access panel configured to allow the tenant's visitor to access the tenant-specific visitor's entry point functionalities associated with the business location of the front desk access panel, and
c. upon completion of a visit with the tenant, operating the front desk access panel to provide the tenant's visitor with the tenant-specific exit point functionalities corresponding to the same business location.

In a further yet aspect of the invention, computer-implemented method for accessing tenant-specific point-of-service visitor functionalities for one of a plurality of locations, each location having one or more business tenants operated thereat, the method may include the following steps:
a. providing each tenant at each business location with a tenant-specific and location-specific access to a multi-tenant computer system to configure tenant-specific and location-specific visitor's functionalities for incoming tenant's visitors to be operated using a location-specific front desk access panel placed at each business location,
b. upon arrival to the business location and self-identification of a visitor using the front desk access panel, operating the computer system to designate the visitor as a tenant's visitor of a specific tenant at that business location,
c. operating the front desk access panel at that location to allow the tenant's visitor to access the tenant-specific visitor's entry point functionalities associated with the tenant's business at that business location, and
d. upon completion of a visit with the tenant, operating the front desk access panel at that location to provide the tenant's visitor with the tenant-specific exit point functionalities corresponding to the same business location.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1 is an exemplary block diagram of the customer/visitor booking process,

FIG. 2 is an exemplary block diagram of the steps that a tenant can take in preparation for the visit by the customer or the client,

FIG. 3 is an exemplary block diagram of the steps that the visitor/customer may take when arriving at the business location,

FIG. 4 is an exemplary block-diagram of the step for the employee clock-in using the same computer system of the present invention, and

FIG. 5 is an exemplary block-diagram of check-in and check-out steps for the visitor suing QR codes to expedite the process.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

In broad terms, the present invention describes a computerized method tailored to enhance the experience of visitors at a business location that accommodates multiple tenants. This method involves several specific steps to ensure that each tenant can offer personalized interactions to their visitors through a central system. Initially, each tenant is given tenant-specific access to a multi-tenant computer system, where they can configure unique visitor functionalities. These functionalities are particularly tailored to each tenant's needs and are designed to be accessed through a front desk panel located at the business premises.

Upon a visitor's arrival, they engage with this front desk access panel by self-identifying themselves, possibly through a digital interface that could include entering their name or scanning a QR code. The computer system recognizes the visitor, designates the person as a guest of a specific tenant, and automatically loads the visitor functionalities that were pre-configured by that tenant. This could include anything from digital sign-in, personalized greetings, to specific instructions or directions within the building.

After the visitor completes their business or appointment with the tenant, they return to the front desk access panel. Here, they can use the exit functionalities provided by the system. These might include confirming the end of the visit, leaving feedback, or even processing any necessary payments. The system could alternatively offer additional services such as scheduling a follow-up appointment or providing digital receipts of the visit.

Each step of this method is designed to streamline the visitor experience at multi-tenant locations, making it more efficient and tailored to individual tenant requirements. By automating both the entry point and exit point processes, the computer system not only saves time but also enhances security and visitor management at multi-tenant business locations.

In other embodiments, a computer-implemented method of the present invention is designed to streamline the process for visitors at a business location that hosts multiple tenants. The method ensures that each visitor's experience is customized to the specific tenant they are visiting. Initially, the method describes steps to grants each tenant unique access to a shared multi-tenant computer system. This access allows tenants to set up specific functionalities tailored to both known and anticipated visitors. These functionalities might include personalized greetings, custom directions within the building, or specific entry point forms that need to be filled out. All these are designed to be accessed via a front desk panel at the business location, ensuring that each tenant can provide a distinct and tailored experience right from the start.

Upon a visitor's arrival at the business location, they may engage with the computer system by self-identifying themselves at the front desk access panel. This could involve entering a name or ID, or scanning a QR code. The computer system may then recognize them as a visitor associated with a particular tenant and sets up the interface accordingly, ensuring that all interactions from this point are customized to the needs and expectations of both the visitor and the tenant.

Once identified, the visitor can utilize various entry point functionalities that have been pre-configured by the tenant they are visiting. These might include digital check-ins, navigating through the facility, or receiving specific information pertinent to their visit. These functionalities are designed to facilitate a smooth and efficient entry process tailored to the visitor's needs.

After the visitor completes their visit, they use the same front desk access panel to engage in the exit functionalities also customized by the tenant. This could include leaving feedback, finalizing any transactions, or confirming the end of the visit. Similar to the entry process, the exit functionalities are designed to be user-friendly and specific to the tenant's requirements, ensuring a seamless close to the visit.

In further embodiments, the computer-implemented method of the invention addresses the issue of managing interactions at a multi-tenant business location by introducing several advanced features and functionalities. The method initially allows tenants to provide the system with unique visitor identifiers beforehand, which the system later uses to confirm the identity of visitors upon arrival, enhancing security and personalization. Additionally, tenants can set expected arrival times for their visitors, and the system will only recognize these visitors if they arrive within the specified window, adding an extra layer of scheduling precision.

As part of the visitor's experience at the business location, specific functionalities may be included to streamline both the check-in and check-out processes. At check-in, the system can handle appointment registrations, while at check-out, it offers options for payments, feedback submission, or scheduling follow-up visits. The method is also equipped to handle walk-in or unanticipated visitors by allowing them to request immediate or future appointments, which accommodates flexibility in visitor management.

For new or anticipated visitors, the system can generate a QR code that links to necessary online forms, ensuring all pertinent data is collected efficiently. It also supports the distribution of promotional materials that are tailored to both the tenant and the location, aiding in targeted marketing efforts. Tenants have the ability to reserve shared resources, such as conference rooms, in line with their scheduled appointments, ensuring that necessary spaces are available without overlap.

Furthermore, the computer system may restrict the activation of certain functionalities to times when the tenant is present at the location, which ensures that the resources and services are utilized correctly and securely. Enhancements like voice recognition technology may be included to improve the ease of use during the interaction process, offering a hands-free alternative that can be particularly beneficial for accessibility. This may be especially beneficial for disabled visitors, or visitors that may have difficultly interacting with a computerized access panel.

Lastly, the method may integrate employee management by enabling tenant employees to clock in at the front desk, which starts tracking their working hours immediately. This feature consolidates employee time management with visitor management, streamlining operations across multi-tenant facilities. These additions make the system highly adaptable, secure, and efficient, catering to a broad range of needs at business locations with multiple tenants.

The invention may use a front desk access panel that may be a front desk computer or a tablet that is accessible to a visitor entering the business location. The front desk access panel may be operatively connected to a server such as a local computer server or a cloud server, which is also accessible to the business tenants housed at the business location. The computer system of the invention may be configured to operate in a single business location housing a number of tenants, or at multiple business locations, each housing one or more business tenants. It may also be configured to operate for a single business tenant that has presence in more than one business location, as the invention in not limited in this regard. The terms “customer”, “client”, and “visitor” are used interchangeably throughout this description.

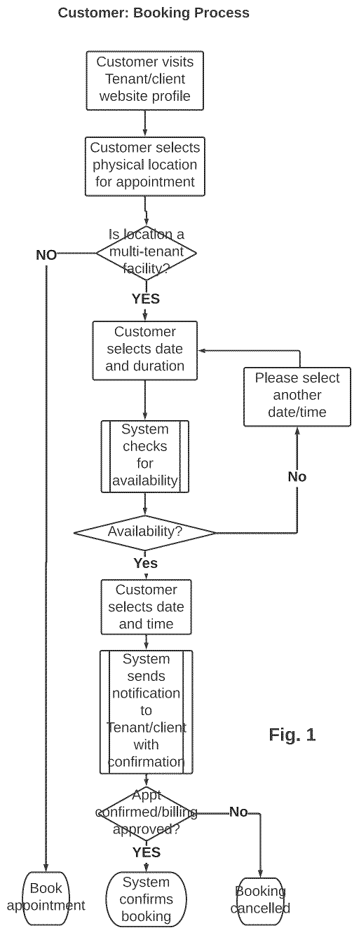

The following description outlines some useful examples of the configuration of the computer system to serve the purposes of managing a visit by an individual to a business location. The flowchart in FIG. 1 outlines one example of a comprehensive customer booking process for securing appointments within a multi-tenant facility using a tenant or client's online profile. Initially, a customer may accesses the designated tenant website to select a physical location where they desire to book an appointment. A critical decision point early in the process determines whether the chosen location operates as a multi-tenant facility. If the location does not meet this criterion, the customer is redirected to revisit and possibly revise their location selection.

Assuming the location qualifies as a multi-tenant facility, the customer proceeds to specify the preferred date and duration for their appointment. This triggers the computer system to verify the availability of the selected time slot. In cases where the desired slot is unavailable, the customer is prompted to choose an alternative date and/or time, effectively looping back to the previous step of date and duration selection until an available slot is found.

Upon successful selection of an available time slot, the customer may finalize their choice of date and time. Subsequently, the system dispatches a notification containing the booking details to the tenant for confirmation. This moves the process to another juncture where the appointment's confirmation and the associated billing details are reviewed for approval. If the tenant does not approve the booking, perhaps due to incorrect or incomplete information, the booking is canceled, and the process could potentially revert to earlier steps allowing the customer to make necessary adjustments and reattempt the booking.

Conversely, if the booking receives approval, the system affirms the reservation, and the booking process concludes successfully. This sequential and interactive procedure ensures that both the customer's requirements and the operational capabilities of the selected business location are adequately addressed, facilitating a streamlined appointment scheduling experience.

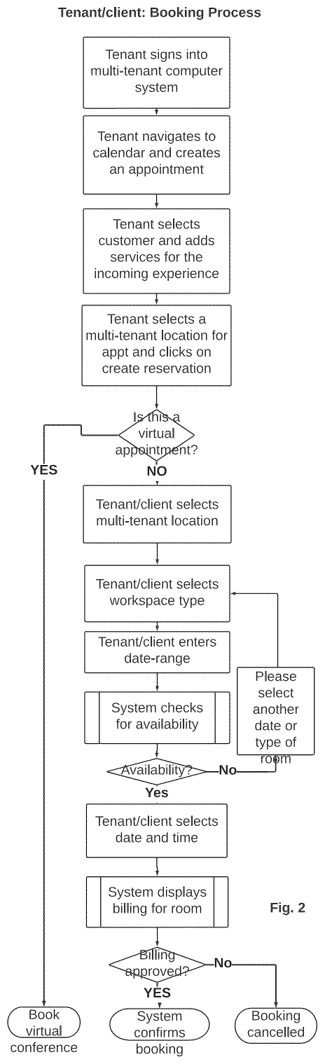

The exemplary flowchart in FIG. 2 describes the booking process from the perspective of a tenant operating at a multi-tenant business location, emphasizing the procedural steps to secure a physical or virtual space for an appointment. The process begins with a tenant logging into a multi-tenant computer system, indicating the desired initiation of an organized workflow designed for booking. Once logged in, the tenant navigates to a calendar within the computer system to create a new appointment for an expected visitor. This step involves specifying the details of the appointment, such as time and purpose, as well as associating the appointment with a specific visitor.

Subsequently, the tenant selects and enters the details about a specific customer for whom the appointment is being scheduled and adds any requisite services that are necessary for the upcoming entry point and/or exit point functionalities. This customization ensures that all required elements are aligned for the appointment. Following the customization of entry point and exit point functionalities, the tenant may decide on the specific location by selecting a particular multi-tenant location and clicking on the option to create a reservation. At this juncture, the computer system may present a decision point: it asks whether the appointment is virtual. If the answer is “Yes,” the process would typically branch into steps specific to setting up a virtual meeting, which are not depicted here. If “No,” the tenant proceeds to select the physical location or a room within the multi-tenant business location.

After selecting the physical space for the visit, the tenant then may choose the type of workspace needed for the appointment, such as a private office, conference room, or shared space. The next step involves entering a date range for when the appointment is intended to take place. The computer system may then check the availability of the selected workspace within the specified date range. If the desired space is not available, the system prompts the tenant to select another date or type of room, looping back to the workspace type selection.

If the space is available, the tenant confirms the date and time. Following this confirmation, the computer system may display the billing information related to the use of the room. A final approval check for billing may take place-if the billing is not approved, the booking is canceled. However, if billing is approved, the computer system may confirm the booking, finalizing the reservation. If the initial inquiry was for a virtual setup and it was confirmed earlier in the process, the tenant may proceed to book the virtual conference at this point, completing the process.

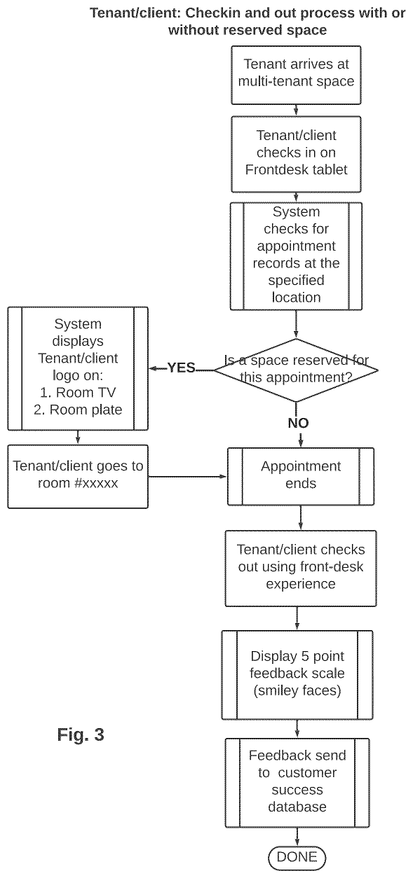

The flowchart in FIG. 3 is key to the present invention. It illustrates an exemplary check-in and check-out procedure for tenants or visitors in a multi-tenant business location, detailing steps for scenarios with and without reserved spaces. The process begins when a tenant or a visitor arrives at a multi-tenant business location. Upon arrival, the individual checks in with self-identification steps using a dedicated computer system access point tablet at the front desk. This initial interaction is crucial as it signifies the tenant's and/or visitor's presence with the facility's management system. The self-identification may be done using a unique visitor-specific identifier. Examples of the unique visitor-specific identifier may include: a visitor's name entered into the front desk access panel, a telephone number entered into the front desk access panel, a visitor-specific barcode entered into the front desk access panel, a computer-recognized visitor-specific identification document entered into the front desk access panel, a computer-operated facial recognition processed by the front desk access panel, or a secure alpha-numerical code entered into the front desk access panel. The computer system at this point, may be operated to compare the entered unique visitor-specific identifier to at least one unique visitor-specific identifier provided by the tenant in step (a) prior to the visit of the visitor.

Furthermore, the computer system may be configured for allowing the entry by the tenant of an expected arrival time period for at least one known tenant's visitor. In this case, upon arrival of the visitor, the computer system may be configured to designate the visitor as a known tenant's visitor only upon arrival thereof during the expected arrival time period.

Following the chart in FIG. 3, the computer system may offer the visitor to go through other specified entry point functionalities that may be selected by the tenant, such as signing a nondisclosure agreement or another appointment check-in steps.

At the completion of the check-in, the computer system may perform an automated check for any appointment records associated with the tenant or visitor at the specified business location. This verification step is essential to match the visitor with the tenant and with the correct records and ensure the appointment's validity and scheduled logistics.

After verifying the appointment details, the computer system may query whether a physical space has been reserved for this specific appointment. If there is a reservation, the computer system may direct the visitor to proceed to the designated room. Guidance to the reserved room may be facilitated through visual aids such as displaying the tenant/client's logo on a room TV or on a room plate, ensuring the visitor navigates directly to the correct location.

If no space has been reserved for the appointment, the process terminates the appointment at this stage. In this scenario, the visitor's engagement with the facility ends without proceeding to any specific room or area, reflecting the dependency of the appointment on prior reservations.

Whether the appointment proceeds in a reserved space or not, the next step involves the visitor checking out at the conclusion of their visit. Checkout is conducted using the same front desk access point tablet, ensuring a full-circle process from check-in to check-out, and maintaining consistency in how interactions are logged within the system. Examples of such exit point functionalities that may be offered to the visitor include the payment for the services, or providing feedback to the tenant. This feedback may be captured via a 5-point feedback scale, represented by smiley faces, ranging from dissatisfied to very satisfied. Collecting feedback at this point is useful for assessing tenant satisfaction and identifying areas for improvement.

Finally, the feedback may be transmitted to the customer success database. This final step concludes the process, ensuring that the visitor's feedback is logged and available for future reference and action by the business location management. This systematized feedback mechanism may plays an important role in ongoing efforts to enhance service quality and tenant satisfaction in the multi-tenant business location.

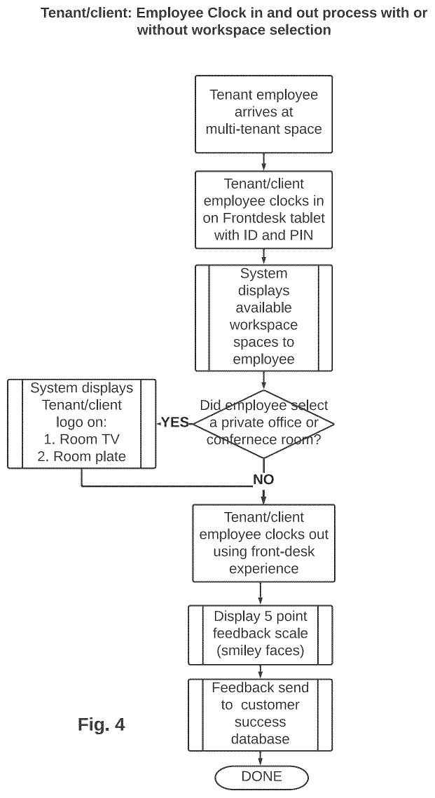

FIG. 4 shows exemplary steps for a walk-in individual who is an employee or another person associated with or working for the tenant at the business location. The computer system may be configured to identify such person from the self-identification data presented by the person during a check-in process. Once checked in, the computer system may present the employee with available workspace options. This message step may allows the employee to choose an appropriate workspace based on availability and their specific needs for the day. This step may be useful for organizing and allocating workspace resources efficiently among various users of the multi-tenant facility.

Following the display of options, the employee may make a choice regarding the type of workspace they require. The decision point in the process queries whether the employee selected a private office or a conference room. If the employee chooses one of these options, the computer system may then confirm the selection and direct the employee to the specific location, facilitated by displaying the tenant/client's logo on the room TV or a digital room plate. This ensures that the employee is guided to the correct reserved area, enhancing the operational flow within the facility.

However, if the employee does not select a private office or conference room—perhaps choosing a more open or communal workspace—or if no selection is made, the process leads directly to the clock-out procedure. In this scenario, the employee's use of specific, reservable workspaces is not documented, reflecting a flexible or non-specific usage pattern.

The final steps occur when the employee is ready to leave the business location. The employee may clock out using the same front-desk access panel tablet used for clocking in, maintaining consistency and security in tracking entry and exit. Upon clocking out, the employee may be prompted to provide feedback on their experience at the business location, utilizing a 5-point feedback scale represented, for example, by smiley faces. This feedback is useful for assessing employee satisfaction and identifying potential areas for improvement in facility management.

Finally, the feedback may be transmitted to the customer success database, concluding the process. This feedback collection is integral to ongoing efforts to enhance the working environment and operational efficiency of the multi-tenant business location, ensuring that it remains responsive to the needs of its diverse users.

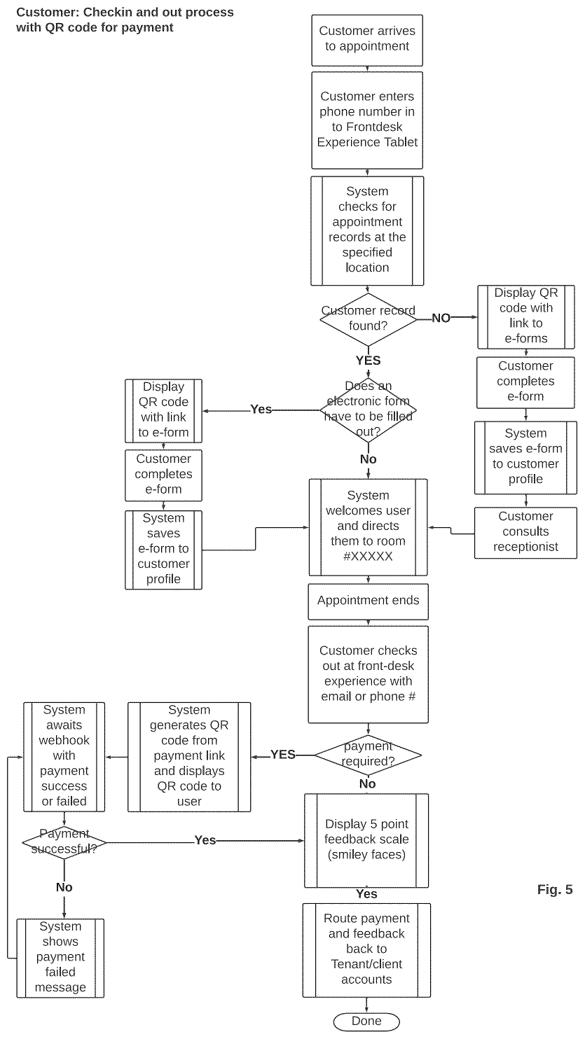

FIG. 5 shows an example of a block diagram showing the check-in and check-out steps for a visitor with an enhancement provided by utilizing QR codes. The flowchart provides a detailed visualization of the check-in and check-out process for customers using a QR code for payments at a multi-tenant facility.

The process begins with the visitor's arrival at the designated appointment location. Upon arrival, the customer checks in by entering their phone number into the front desk access panel such as an experience tablet. This step helps the computer system to identify the customer and retrieve any relevant appointment records at the specified location. As explained above, the self-identification of the visitor may be done using a unique visitor-specific identifier, such as a phone number.

Once the phone number is entered, the system searches for the customer's appointment record. If no record is found, the process ends, and presumably, the customer would need to consult the receptionist or call the tenant to rectify the issue through other means. If a record is found, the system then checks if there is an electronic form that needs to be filled out as part of the appointment process. Alternatively, other entry point functionalities may be offered to the visitor.

If an electronic form is required, the system may display a QR code with a link to the e-form. The customer may then scan the QR code, complete the e-form, and upon submission, the computer system may save the completed form to the customer's profile. If no form is required, the computer system may directly welcome the customer and direct them to their designated room, identified by a specific room number, allowing the appointment to proceed.

After the completion of the appointment, the visitor checks out at the front-desk by again using either their email or phone number. At this stage, the computer system may determine whether a payment is required for the services received. Payment is just one example of an exit point functionality that may be specified by the tenant for the completion of the visit. If no payment is needed, the computer system may then prompt the customer to provide feedback, such as using a 5-point smiley face scale, gathering valuable insights into the customer experience.

However, if payment is required, the computer system may generate a QR code from a payment link and display this QR code to the visitor. The visitor may then scan the QR code to make a payment. The computer system awaits confirmation of payment success via a webhook. If the payment is successful, the feedback scale may be displayed, and both payment and feedback information are routed back to the tenant/client accounts for processing and records.

If the payment fails, the system may display a “payment failed” message to the customer, potentially requiring further action to complete the transaction. This comprehensive process ensures that all aspects of the customer experience, from check-in to payment and feedback, are efficiently managed and recorded, enhancing customer satisfaction and operational efficacy.

The use of QR codes may allow the entire computer system to be off-line and not directly connected to the Internet, which increases the safety of the computer system and minimizes a chance of a hacker attack.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A computer-implemented method for accessing tenant-specific point-of-service visitor functionalities associated with a specific multi-tenant business location, the method comprising the following steps:
a. providing each tenant with a tenant-specific access to a multi-tenant computer system to configure tenant-specific and location-specific visitor's functionalities to be operated using a front desk access panel at the specific business location,
b. upon arrival and self-identification of a tenant's visitor to the specific business location, providing the tenant's visitor with the front desk access panel configured to allow the tenant's visitor to access the tenant-specific visitor's entry point functionalities associated with the business location of the front desk access panel, and
c. upon completion of a visit with the tenant, operating the front desk access panel to provide the tenant's visitor with the tenant-specific exit point functionalities corresponding to the same business location.

2. A computer-implemented method for accessing tenant-specific point-of-service visitor functionalities associated with a specific multi-tenant business location housing a plurality of business tenants, the method comprising the following steps:
a. providing each tenant with a tenant-specific access to a multi-tenant computer system to configure tenant-specific and location-specific visitor's functionalities for known and anticipated tenant's visitors to be operated using a front desk access panel at the specific business location,
b. upon arrival to the business location and self-identification of a visitor using the front desk access panel, operating the computer system to recognize and designate the visitor as a known tenant's visitor of a specific tenant at the business location,
c. operating the front desk access panel to allow the tenant's visitor to access the tenant-specific visitor's entry point functionalities associated with the tenant's business at the business location, and
d. upon completion of a visit with the tenant, operating the front desk access panel to provide the tenant's visitor with the tenant-specific exit point functionalities corresponding to the same business location.

3. The computer-implemented method, as in claim 2, wherein in step (b), the self-identification of the visitor is done using a unique visitor-specific identifier.

4. The computer-implemented method, as in claim 3, wherein the unique visitor-specific identifier is selected from a group consisting of: a visitor's name entered into the front desk access panel, a telephone number entered into the front desk access panel, a visitor-specific barcode entered into the front desk access panel, a computer-recognized visitor-specific identification document entered into the front desk access panel, a computer-operated facial recognition processed by the front desk access panel, or a secure alpha-numerical code entered into the front desk access panel.

5. The computer-implemented method, as in claim 3, wherein in step (b), the computer system is operated to compare the entered unique visitor-specific identifier to at least one unique visitor-specific identifier provided by the tenant in step (a) prior to the visit of the visitor.

6. The computer-implemented method, as in claim 2, wherein in step (a), the computer system is configured for entry by the tenant of an expected arrival time period for at least one known tenant's visitor, wherein in step (b) the computer system is configured to designate the visitor as a known tenant's visitor only upon arrival thereof during the expected arrival time period.

7. The computer-implemented method, as in claim 2, wherein in step (c) the tenant-specific entry point functionality is an appointment check-in.

8. The computer-implemented method, as in claim 2, wherein in step (d) the tenant-specific exit point functionality is at least one of: a payment interface, a feedback interface, or a follow-up interface.

9. The computer-implemented method, as in claim 2, wherein in step (b), the computer system is further configured to allow an unknown visitor to request an immediate or a scheduled visit with the specific tenant housed at the business location.

10. The computer-implemented method, as in claim 9, wherein in step (b), the computer system is further configured to supply a new visitor with a barcode or a QR code pointing to an online location of one or more fillable forms preconfigured by the tenant for use by new visitors.

11. The computer-implemented method, as in claim 9, wherein in step (b), the computer system is further configured to provide an unknown visitor with a promotional material preconfigured by the tenant for new visitors.

12. The computer-implemented method, as in claim 11, wherein in step (b), the promotional material is both tenant-specific and location-specific to the business location.

13. The computer-implemented method, as in claim 2, wherein in step (a), the computer system is further configured to allow the tenant to reserve a common shared resource at the business location for the time period of an anticipated visit.

14. The computer-implemented method, as in claim 13, wherein the common shared resource is a conference room at the business location.

15. The computer-implemented method, as in claim 2, wherein steps (c) or (d) are implemented only during the time when the tenant is known to have access to or known to be present at the business location.

16. The computer-implemented method, as in claim 2, wherein at least one of the steps (b), (c), or (d) are implemented using a voice recognition option, thereby improving ease of use by the visitor.

17. The computer-implemented method, as in claim 2, wherein in step (b) after self-identification of the visitor, the computer system is further configured to recognize the visitor as an employee of or a person associated with a specific tenant, wherein in step (c), the operating of the front desk access panel is adjusted to allow the employee or a person associated with a specific tenant to clock in and start counting working hours therefor.

18. A computer-implemented method for accessing tenant-specific point-of-service visitor functionalities for one of a plurality of locations, each location having one or more business tenants operated thereat, the method comprising the following steps:
a. providing each tenant at each business location with a tenant-specific and location-specific access to a multi-tenant computer system to configure tenant-specific and location-specific visitor's functionalities for incoming tenant's visitors to be operated using a location-specific front desk access panel placed at each business location,
b. upon arrival to the business location and self-identification of a visitor using the front desk access panel, operating the computer system to designate the visitor as a tenant's visitor of a specific tenant at that business location,
c. operating the front desk access panel at that location to allow the tenant's visitor to access the tenant-specific visitor's entry point functionalities associated with the tenant's business at that business location, and
d. upon completion of a visit with the tenant, operating the front desk access panel at that location to provide the tenant's visitor with the tenant-specific exit point functionalities corresponding to the same business location.
